(19) United States
(12) Patent Application Publication
(10) Pub. No.: US 2004/0059140 A1
(43) Pub. Date: Mar. 25, 2004

(54) 14(15)-unsaturated 15- and/or 16-substituted androgens with mixed androgen-progrestational profile

(76) Inventors: Japp Van Der Louw, Oss (NL); Dirk Leysen, Oss (NL); Marcel Evert De Gooyer, Oss (NL)

Correspondence Address: William M Blackstone, Akzo Nobel Patent Department Intervet Inc, 405 State Street, Millsboro, DE, 19966, US

(21) Appl. No.: 10/450,693

(22) PCT Filed: Dec. 10, 2001
(86) PCT No.: PCT/EP01/14776

(30) Foreign Application Priority Data
Dec. 15, 2000 (EP) 00204543.3

(51) Int. Cl. C07J 41/00; C07J 7/00

(52) U.S. Cl. 552/519; 552/592

(57) ABSTRACT

Disclosed are androgen-related steroids having a Δ14 double bond and carrying a substituent at either or both of the carbon atoms nos. 15 and 16. The present steroids are characterized by possessing a mixed profile of androgenic and progestagenic activities. This makes these compounds suitable for male or female hormone replacement therapy, as well as male contraception.

The invention is in the field of synthetic hormones having a mixed profile of androgenic and progestagenic activities.

Due to modern demands for hormone replacement therapy (HRT) in males and females, as well as the upcoming issue of male contraception, a renewed interest exists in androgens. Focus therewith is on improvements such as oral activity, a high potency, or both. See, e.g. WO 99/67271, WO 00/53619, WO 00/59920, and EC 2000-3572 (non-prepublished patent application PCT/EP00/06544).

A known potent androgen is the so-called “Segaloff steroid” which is a 19-nortestosterone derivative having a 7α-methyl group, and which has a double bond between carbon atoms 14 and 15 (Δ¹⁴). This steroid has long been recognized as the most potent oral androgen known. See, int.al. Avery et al, Steroids, 55, 59 (1990). The compound, together with its 7α-H analogue, is also known from GB 1,341,601. The compounds disclosed in PCT/EP00/06544 display a major improvement as compared to Segaloff in that they are better suitable for clinical use, and particularly possess sufficient oral activity and metabolic stability.

The present invention has its focus on an entirely different type of androgens, viz. those having a nuxed profile of androgenic and progestagenic activities (i.e. compounds which possess both activities intrinsically united within one molecule, and preferably well balanced). Compounds having such a profile are desired for use in HRT, as well as for male contraception, for which they have the advantage that they do not require the separate administration of a progestagen.

Surprisingly, such mixed androgenic-progestagenic steroids (A/P steroids) were found within the group of Δ¹⁴ steroids. These compounds according to the invention are characterized by satisfying the general structural formula I given below.

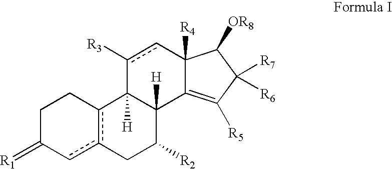

wherein

R₁ is O, (H,H), (H,OR), NOR, with R being hydrogen, (C₁₋₆) alkyl, or (C₁₋₆) acyl;

R₂ is hydrogen, (C₁₋₄) alkyl, or (C₂₋₄) alkenyl;

R₃ is hydrogen, (C₁₋₄) alkyl, or (C₂₋₄) alkenyl; preferred is hydrogen or β-methyl;

R₄ is (C₁₋₂) alkyl;

R₅ is hydrogen, (C₁₋₄) alkyl, (C₂₋₄) alkenyl;

R₆ and R₇ are independently hydrogen, (C₁₋₄) alkyl, or (C₂₋₄) alkenyl;

R₈ is hydrogen, or (C₁₋₁₅) acyl;

and the dotted lines indicate optional bonds, and wherein at least one of R₅, R₆ and R₇ is not hydrogen.

Particularly preferred in compounds wherein R₃ is hydrogen or β-methyl is to select methyl for R₄. In general, preferred compounds of the invention are compounds in which R₁ is oxo, R₃ is hydrogen, R₄ is methyl, and the dotted lines indicate a Δ⁴ double bond, whereby it is prefered in this group that R₅ or R₆ is methyl and more preferred R₆ is methyl. For the latter group (R₆ is methyl) it is recommended to select for R₂ methyl, ethyl or vinyl.

Even more preferred are compounds in which R₂ is selected from the group consisting of methyl, ethyl, and ethenyl, either or both of R₅ and R₆ are methyl and R₇ is hydrogen. Specifically preferred compounds of the invention are (7α,17β)-17-hydroxy-7,15-dimethylestra-4,14-dien-3-one and (7α,16α,17β)-17-hydroxy-7,16-dimethylestra-4,14-dien-3-one.

The term (C₁₋₆) alkyl as used in the definition of formula I means a branched or unbranched alkyl group having 1-6 carbon atoms, like methyl, ethyl, propyl, isopropyl, butyl, isobutyl, tertiary butyl, pentyl, and hexyl. Likewise, the term (C₁₋₄) alkyl means a branched or unbranched alkyl group having 1-4 carbon atoms and the term (C₁₋₂) alkyl means an alkyl group having 1-2 carbon atoms.

The term (C₂₋₄) alkenyl means a branched or unbranched alkenyl group having at least one double bond and 2-4 carbon atoms. Preferred alkenyl groups have 2-3 carbon atoms, such as vinyl and propenyl.

The term (C₂₋₄) alkynyl means a branched or unbranched alkynyl group having at least one triple bond and 2-4 carbon atoms. Preferred alkynyl groups have 2-3 carbon atoms, such as ethynyl and propynyl.

The term (C₁₋₄) alkylidene means a branched or unbranched alkylidene group having 1-4 carbon atoms. Preferred alkylidene groups have 1-2 carbon atoms, and most preferred is methylene.

The term (C₂₋₄) alkenylidene means a branched or unbranched alkenylidene group having 2-4 carbon atoms. Preferred alkenylidene groups have 2-3 carbon atoms, such as ethenylidene.

The term (C₃₋₆) cycloalkyl or (C₃₋₆) cycloalkane ring means a cycloalkane ring having 3-6 carbon atoms, like cyclopropane, cyclobutane, cyclopentane and cyclohexane.

The term (C₅₋₆) cycloalkenyl or (C₅₋₆) cycloalkene ring means a cycloalkene ring having, at least one double bond and 5 or 6 carbon atoms.

The term (C₁₋₆) acyl means an acyl group derived from a carboxylic acid having 1-6 carbon atoms, like formyl, acetyl, propanoyl, butyryl, 2-methylpropanoyl, pentanoyl, pivaloyl, and hexanoyl. Likewise, the term (C₁₋₁₅) acyl means an acyl group derived from a carboxylic acid having 1-15 carbon atoms. Also included within the definition of (C₁₋₆) acyl or (C₁₋₁₅) acyl are acyl groups derived from dicarboxylic acids, like hemi-maloyl, hemi-succinoyl, hemi-glutaroyl, and so on. Preferred is hemi-succinoyl.

The term halogen means fluorine, chlorine, bromine, or iodine. When halogen is a substituent at an alkyl group, Cl and F are preferred, F being most preferred.

It is understood that the 15-substituted and/or 16-substituted Δ¹⁴-nandrolone derivatives of the invention have the natural configurations 5α, 8β, 9α, 10β, 13β, and 17β.

The 15-substituted and/or 16-substituted Δ¹⁴-nandrolone derivatives of this invention have the natural configurations 5α, 8β, 9α, 10β, 13β and 17β, and may possess also one or more additional chiral carbon atoms. The compounds may therefore be obtained as a pure diastereomer, or as a mixture of diastereomers. Methods for obtaining the pure diastereomers are well known in the art, e.g. crystallization or chromatography.

The compounds of the invention, which are distinguished from the aforementioned known “Segaloff steroid,” and from those described in PCT/EP00/06544, by the presence of a 15-substituent and/or 16-substituent, possess a mixed androgen/progestational profile.

The compounds of the invention as described hereinbefore in general possess an unexpected combination of androgenic and progestagenic activity. Androgenic activity can be measured in various ways. Thus, the potency of androgens can be determined in vitro using the cytoplasmic androgen receptor from human breast tumor cells (MCF-7 cell line); see Bergink, E. W. et al, Comparison of the receptor binding properties of nandrolone and testosterone under in vitro and in vivo conditions, J. Steroid Biochem. 22, 831-836 (1985). It is also possible to use Chinese hamster ovary (CHO) cells transfected with the human androgen receptor (incubation time 16 h, temperature 4° C.) and compared with the affinity of 5α-dihydrotestosterone [according to the procedure described by Bergink, E. W. et al, J. Steroid Biochem. 19, 1563-1570 (1983)]. The transactivative androgen activity of the compounds of the invention can be measured, e.g. in Chinese hamster ovary cells (CHO) transfected with the human androgen receptor (hAR), in combination with a mouse mammary tumor virus (MMTV), and luciferase receptor gene (incubation time 16 h, temperature 37° C.) and compared with the activity of 5α-dihydrotestosterone [according to the procedure described by Schoonen, W. G. E. J. et al, Analyt. Biochem. 261, 222-224 (1998)]. For the in vivo potency determination of androgens the classical Hershberger test can be used. In this test the androgenic (increase in prostate weight) and anabolic activities [increase of the musculus levator ani (MLA)] of a compound are tested in immature castrated rats after daily administration for 7 days; see Hershberger, L. G. et al, Myotrophic activity of 19-Nortestosterone and other steroids determined by modified levator ani muscle method, Proceedings of the society for experimental biology and medicine 83, 175-180 (1953). Additionally, the effect of an androgenic compound on LH suppression can be tested in mature castrated rats according to Kumar, N. et al, The biological activity of 7alpha-methyl-19-nortestosteione is not amplified in male reproductive tract as is that of testosterone, Endocrinology 130, 3677-3683 (1992).

The transactivative progestagenic activity of the compounds of the invention can be measured, e.g. in Chinese hamster ovary cells (CHO) transfected with the human progesterone receptor B (hPRB), in combination with a mouse mammary tumor virus (MMTV), and luciferase receptor gene (incubation time 16 h, temperature 37° C.) and compared with the activity of (16α)-16-ethyl-21-hydroxy-19-norpregn-4-ene-3,20-dione [according to the procedure described by Schoonen, W. G. E. J. et al, Analyt. Biochem. 261, 222-224 (1998)].

As mixed androgenic/progestagenic hormones the Δ¹⁴ steroids of the present invention can be used in, int.al., male contraception and male HRT (hormone replacement therapy). Thus, e.g. male contraception frequently is described to comprise a regimen of administration of hormones in which a progestagen serves to achieve a contraceptive effect and an androgen serves to supplement the resulting decreased testosterone level. Although the present compounds could be used as either the progestagen or the androgen, and be supplemented with yet another compound having the other activity, the A/P nature of the the Δ¹⁴ steroids of the present invention opens up the possibility to achieve male contraception through the progestagen-androgen system, on the basis of one single compound.

The A/P compounds of the invention can also be used for androgen supplementation in the partially androgen deficient ageing male. The progestagenic activity leads to an advantage of the compounds of the invention in that the production of endogenic testosterone is suppressed. This opens up the possibility to supplement the resulting deficiency by the administration of a selected exogeruic androgen, which is safer than testosterone. Testosterone is converted by 5α-reductase to the more potent 5α-dihydro-testosterone. Well-known detrimental effects on sites where a relatively high concentration 5α-reductase is present, are prostate problems, acne, hair-loss. Furthermore, irrespective of the 5α-reduction, exogenic testosterone per se is safer for the prostate than endogenic testosterone, due to lower concentrations present in the prostate. Hence, by virtue of the A/P compounds of the present invention, one can advantageously diminish the intrinsic detrimental effects of endogenic testosterone, and administer an androgen which is not 5α reducable, such as MENT, or the androgens disclosed in WO 99/67271, WO 00/53619, WO 00/59920, and EC 2000-3572.

Next to the use in the male, the compounds of the invention also can be used in the female, e.g. as androgen/progestagen replacement therapy in postmenopausal women. Here the the steroids of the invention have the advantage of a progestagenic component, which has a positive effect on the endometrium (inhibition of estrogen-induced proliferation).

The present invention also relates to a pharmaceutical composition comprising a steroid compound according to the invention mixed with a pharmaceutically acceptable auxiliary, such as described in the standard reference, Gennaro et al, Remmington's Pharmaceutical Sciences, (18th ed., Mack Publishing Company, 1990, see especially Part 8: Pharmaceutical Preparations and Their Manufacture). The mixture of the steroid compounds according to the invention and the pharmaceutically acceptable auxiliary may be compressed into solid dosage units, such as pills, tablets, or be processed into capsules or suppositories. By means of pharmaceutically suitable liquids the compounds can also be applied as an injection preparation in the form of a solution, suspension, emulsion, or as a spray, e.g. nasal spray. For making dosage units, e.g. tablets, the use of conventional additives such as fillers, colorants, polymeric binders and the like is contemplated. In general any pharmaceutically acceptable additive which does not interfere with the function of the active compounds can be used. The steroid compounds of the invention may also be included in an implant, a vaginal ring, a patch, a gel, and any other preparation for sustained release.

Suitable carriers with which the compositions can be administered include lactose, starch, cellulose derivatives and the like, or mixtures thereof used in suitable amounts.

Furthermore, the invention relates to the use of the steroid compound according to the invention for the manufacture of a medicament in the treatment of androgen-deficiency, progestagen-deficiency, and particularly androgen/progestagen deficiency, such as in male or female HRT (hormone replacement therapy). Accordingly, the invention also includes a method of treatment in the field of male or female HRT, comprising the administration to a male or female patient suffering from any of the above-mentioned deficiencies, of a compound as described hereinbefore (in a suitable pharmaceutical dosage form). As has been made clear above, also in male HRT the progestagenic component of the present androgens can be utilized with advantage.

Further, the invention relates to the use of a steroid compound according to the invention for the manufacture of a medicament having contraceptive activity (for which in the art the term “contraceptive agent” is also used). Thus the invention also pertains to the medical indication of contraception, i.e. a method of contraception comprising the administration to a subject, being a male, preferably a human male, of a compound as described hereinbefore (in a suitable pharmaceutical dosage form), preferably as a single active agent.

The A/P compounds according to the invention can also be used in a kit for male contraception. Although, in addition to means for the administration of an Δ¹⁴ A/P steroid according to the invention, this kit can comprise means for the administration of an androgen and/or means for the administration of a progestagen, it is preferred that it serves to administer as the single active agent the Δ¹⁴ A/P steroid of the invention. The means for administering any of the compounds referred to is a pharmaceutical formulation comprising the relevant compound, and a pharmaceutically acceptable carrier.

The invention also pertains to a method of treatment comprising administering to a (notably human) male or female in need of androgen-supplementation, progestagen-supplementation, or (particularly) androgen-progestagen supplementation, a therapeutically effective amount of a 15-substituted, 16-substituted, or 15 and 16 substituted Δ¹⁴ steroid as described hereinbefore. This is irrespective of whether or not the need for androgen-supplementation has arisen as a result of male contraception involving the administration of an additional sterilitant, such as a separate progestagen.

Further, the invention pertains to a method of contraception, comprising administering to a fertile male, notably human, a 15-substituted, 16-substituted, or 15 and 16 substituted Δ¹⁴ steroid as described hereinbefore in a dosage amount and regimen which is sufficient for said compound to be contraceptively effective per se and simultaneously serves to maintain a sufficient androgen-level in the male subject to this contreaceptive method. Alternatively, the method of contraception provided by the present invention comprises administering to a fertile male, notably human, a contraceptively effective combination of a sterilitant, such as a progestagen, and a Δ¹⁴ steroid as described hereinbefore. As a second alternative, the method of contraception involves the administration of a compound according to the invention as the (progestagenic) sterilitant, wherein the maintenance of a sufficient androgen-level is taken care of, in part, by the androgen-component of the activity of the Δ¹⁴ steroid of the invention, and is supplemented by an additional androgen.

The compounds of the invention may be produced by various methods known in the art of organic chemistry in general, and especially in the art of the chemistry of steroids (see, for example: Fried, J. et al, Organic Reactions in Steroid Chemistry, Volumes I and II, Van Nostrand Reinhold Company, New York, 1972).

Convenient starting materials for the preparation of 15-substituted and/or 16-substituted compounds of formula I wherein R₁ is oxo, R₂, R₄, R₅, R₆ and R₇ have the previously given meaning, R₃ is hydrogen or (C₁₋₄) alkyl, R₈ is hydrogen, and the dotted lines indicate a Δ⁴ double bond, are for instance 3-methoxygona-1,3,5(10),15-tetraen-17-one derivatives of general formula II and 3-methoxygona-1,3,5(10),14-tetraen-17-one derivatives of general formula III, wherein R₂ and R₄ have the previously given meaning and R₃ is hydrogen or (C₁₋₄) alkyl, whose synthesis is known in literature, or which can be prepared using standard methods (see e.g. WO 00/53619).

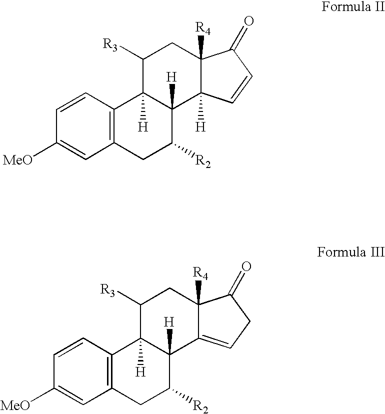

A possible synthesis route for 15-substituted compounds of the invention is as follows. Conjugated addition (e.g. Cu-catalyzed) of an (organometallic) compound of formula R₅M wherein R₅ has the previously given meaning, and M is Li, MgX, ZnX, CeX₂, SiR₃ or SnR₃, to a compound of formula II provides a 15β-substituted 3-methoxygona-1,3,5(10)-trien-17-one derivative. Introduction of a Δ¹⁵ double bond, for instance by conversion to the enol silyl ether and reaction with a Pd(II) salt [Bull, J. R. et al, J. Chem. Soc., Perkin Tr. I, 1269 (1996)] and acid-catalyzed isomerization [Ponsold, K. et al, J. Prakt. Chem. 323, 819 (1981)] produces a 15-substituted 3-methoxygona-1,3,5(10),14-tetraen-17-one derivative. The latter is reduced to the corresponding 17β-hydroxy compound whereafter Birch reduction of the aromatic ring [Caine, D. in Org. Reactions 23, p. 1, Wiley, New York, 1976] and hydrolysis of the resulting 15-substituted (17β)-3-methoxygona-2,5(10),14-trien-17-ol then provides a 15-substituted (17β)-17-hydroxygona-4,14-dien-3-one derivative of the invention.

A possible synthesis route for 16-substituted compounds of the invention is as follows. Alkylation of compounds of formula III results in 16α-substituted and/or 16β-substituted 3-methoxygona-1,3,5(10),14-tetraen-17-one derivatives. Optionally, these compounds can be used as starting material in a second alkylation reaction to afford 16,16-dialkylated 3-methoxygona-1,3,5(10),14-tetraen-17-one derivatives. Reduction of the carbonyl group at C-17, Birch reduction and hydrolysis then affords a 16-substituted or 16,16-disubstituted (17β)-17-hydroxygona-4,14-dien-3-one compounds of the invention.

Compounds of the invention, substituted at C-15 and C-16, are obtained by alkylation at C-16 of 15-substituted 3-methoxygona-1,3,5(10),14-tetraen-17-one derivatives as described above.

Compounds of the invention in which R₁ is (H,H), (H,OR), NOR, with R being hydrogen, (C₁₋₆) alkyl, (C₁₋₆) acyl, are obtained, by using methods known in the art, from compounds of formula I in which R₁ is oxo.

Compounds with substituents R₃ other than hydrogen or (C₁₋₄) alkyl can be obtained from e.g. (11β)-11-(hydroxymethyl)-3-methoxyestra-1,3,5(10)-trien-17-one cyclic 1,2-ethanediyl acetal [van den Broek, A. J. et al, Steroids 30, 481 (1977)], 3-methoxyestra-1,3,5(10)-triene-11,17-dione cyclic 17-(1,2-ethanediyl acetal) [van den Broek, A. J. et al, Recl. Trav. Chim. Pays-Bas 94, 35 (1975)], or (7α)-7-methylestr-4-ene-3,11,17-trione cyclic 3-(1,2-ethanediyl dithioacetal) (WO 94/18224).

Compounds in which R₄ is e.g. ethyl can be prepared from e.g. 13-ethylgon-4-ene-3,17-dione [Brito, M. et al, Synth. Comm. 26, 623 (1996)] or from (7α,17β)-13-ethyl-3-methoxy-7-methylgona-1,3,5(10)-trien-17-ol [FRAD 87961 (1966)].

Compounds of the invention in which R₈ is (C₁₋₁₅) acyl are obtained from compounds of formula I in which R₈ is hydrogen.

Compounds of the invention in which the dotted lines indicate a Δ⁵⁽¹⁰⁾ double bond are produced from the Δ^{2,5(10)} dienes obtained after the Birch reduction. Alternatively, they can be prepared from Δ⁴ derivatives by isomerization.

5α-Reduced compounds of the invention are produced from Δ⁴ derivatives.

Compounds having a Δ¹¹ double bond can be prepared from e.g. estra-4,11-diene-3,17-dione [Broess, A. I. A. et al, Steroids 57, 514 (1992)].

The invention will be further explained hereinafter with reference to the following Examples.

### EXAMPLE 1

(7α,17β)-17-Hydroxy-7,15-dimethylestra-4,14-dien-3-one.

i)—A solution of (7α)-3-methoxy-7-methylestra-1,3,5(10),15-tetraen-17-one [Rasmusson, G. H. et al, Steroids 22, 107 (1973); 10.0 g] in dry tetrahydrofuran (493.2 ml), containing copper(II) acetate (3.38 g), was cooled to −20° C. Methylmagnesium chloride (3 M solution in tetrahydrofuran, 83.1 ml) was added dropwise and the reaction mixture was stirred for 1 h. Then it was poured into a saturated aqueous solution of ammonium chloride and the product was extracted into ethyl acetate. The combined organic phases were washed with a saturated aqueous solution of ammonium chloride and brine, dried over sodium sulfate and concentrated under reduced pressure, to give (7α,15β)-3-methoxy-7,15-dimethylestra-1,3,5(10)-trien-17-one (10.77 g). The product was used in the following step without further purification.

ii)—A solution of diisopropylamine (6.27 ml) in dry tetrahydrofuran (37 ml) was cooled to −30° C. n-BuLi (1.6 M solution in hexanes, 25.3 ml) was added dropwise and stirring was continued at −10° C. for 10 min. The reaction mixture was cooled to −75° C. and a solution of the product obtained in the previous step (3.85 g) in dry tetrahydrofuran (58 ml) was added dropwise. Stirring was continued for 1 h. Chlorotrimethylsilane (4.7 ml) was added and the temperature was allowed to raise to room temperature. The mixture was stirred for 20 min., recooled to 0° C. and then quenched with a saturated aqueous solution of ammonium chloride (123 ml). The product was extracted into ethyl acetate; the combined organic phases were washed with brine, dried over sodium sulfate and concentrated under reduced pressure, to give (7α,15β)-3-methoxy-7,15-dimethyl-17-[(trimethylsilyl)oxy]estra-1,3,5(10),16-tetraene (5.45 g). The product was used in the following step without further purification.

iii)—A solution of the product obtained in the previous step (5.11 g) in acetonitril (128 ml), containing palladium(II) acetate (3.0 g) was heated under reflux for 15 min. The reaction mixture was cooled, filtered and concentrated under reduced pressure, to give (7α)-3-methoxy-7,15-dimethylestra-1,3,5(10),15-tetraen-17-one (5.56 g). The product was used in the following step without further purification.

iv)—A solution of the product obtained in the previous step (3.74 g) in dry toluene (143 ml), containing pyridiniump-toluenesulfonate (2.1 g), was heated under reflux for 15 min. After cooling, the reaction mixture was poured into a saturated aqueous solution of sodium hydrogencarbonate and the product was extracted into ethyl acetate. The combined organic phases were washed with brine, dried over sodium sulfate and concentrated under reduced pressure. Column chromatography afforded (7α)-3-methoxy-7,15-dimethylestra-1,3,5(10),14-tetraen-17-one (1.80 g). The product was used in the following step without further purification.

v)—A solution of the product obtained in the previous step (0.60 g) in dry tetrahydrofuran (17 ml) was added dropwise to a suspension of lithium aluminium hydride (0.291 g) in tetrahydrofiran (17 ml), cooled to 0° C. After stirring of the mixture for 1 h, the reaction was quenched by addition of a saturated aqueous solution of sodium sulfate. The reaction mixture was filtered over dicalite and the filtrate was concentrated under reduced pressure, to give (7α,17β)-3-methoxy-7,15-dimethylestra-1,3,5(10),14-tetraen-17-ol (0.61 g). The product was used in the following step without further purification.

vi)—The alcohol obtained in the previous step (0.61 g) in dry tetrahydrofuran (22 ml) was added to a refluxing solution of lithium (1.4 g) in liquid ammonia (155 ml). After 2 h stirring, tert-butanol (12 ml) was added and stirring was continued for 30 min. Ethanol was added and the ammonia was allowed to evaporate. Water was added and the product was extracted into ethyl acetate. The combined organic phases were washed with water and brine, dried over sodium sulfate and concentrated under reduced pressure, to give (7α,17β)-3-methoxy-7,15-dimethylestra-2,5(10),14-triene-17-ol (0.73 g). The product was used in the following step without further purification.

vii)—A solution of the product obtained in the previous step (0.73 g) in acetone (41 ml) was treated with hydrochloric acid (12 M, 2.07 ml). After 1 h stirring at room temperature, the reaction mixture was poured into water and the product was extracted into ethyl acetate. The combined organic phases were washed with a saturated aqueous solution of sodium hydrogencarbonate and brine, dried over sodium sulfate and concentrated under reduced pressure. Column chromatography afforded (7α,17β)-17-hydroxy-7,15-dimethylestra-4,14-dien-3-one (0.32 g), m.p. 164-167° C., ¹H NMR (CDCl₃) δ 5.85 (bs, 1H), 3.86 (m, 1H), 1.73 (bs, 3H), 0.97 (s, 3H), 0.87 (d, 3H, J 7.1 Hz).

### EXAMPLE 2

(7α,17β)-15-Ethyl-17-hydroxy-7-methylestra-4,14-dien-3-one.

The title compound was prepared from (7α)-3-metlioxy-7-methylestra-1,3,5(10),15-tetraen-17-one [Rasmusson, G. H. et al, Steroids 22, 107 (1973)] following procedures analogous to those described in Example 1. ¹H NoM (CDCl₃) δ 5.85 (bs, 1H), 3.85 (m, 1H), 2.67 (m, 1H), 0.98 (t, 3H, J=7.5 Hz), 0.97 (s, 3H), 0.85 (d, 3H, J=7.5 Hz).

### EXAMPLE 3

(7α,17β)-7-Ethyl-17-hydroxy-15-methylestra-4,14-dien-3-one.

i)—Chlorotrimethylsilane (422.4 ml) was added in 15 min. to a suspension of (17α)-17-hydroxy-19-norpregna-4,6-dien-20-yn-3-one (Syntex S. A., GB 935116 (1958); 400 g in a mixture of dichloromethane (1600 ml) and pyridine (555.2 ml), cooled to 0° C. After 2 h stirring at, 0° C. the reaction mixture was poured into a saturated aqueous solution of ammonium chloride. The product was extracted into dichloromethane; the combined organic phases were washed with water and brine, dried over sodium sulfate and concentrated under reduced pressure, to afford (17α)-17-[(trimethylsilyl)oxy]-19-norpregna-4,6-dien-20-yn-3-one (492 g). The product was used in the following step without further purification.

ii)—A mixture of lithium (18.9 g) and dry diethyl ether (1175 ml) was cooled to −30° C. A solution of bromoethane (101.7 ml) in dry diethyl ether (200 ml) was added in 1 h (T<−20° C.) and the reaction mixture was stirred at −25° C. for 1 h. In a second flask, a suspension of copper(I) iodide (115.5 g) in dry tetrahydrofuran (1763 ml) was cooled to −40° C. Dry lithiumbromide (160.7 g) was added and the mixture was stirred for 5 min. The solution of ethyllithium was added dropwise in 1 h (−40<T<−30° C.) and the resulting cuprate solution was stirred for 30 min. at −30° C. A solution of the product obtained in the previous step (114.8 g) in dry tetrahydrofuran (1763 ml) was added dropwise (−30<T<−25° C.). After 1 h stirring at that temperature, chlorotrimethylsilane (113 ml) was added (T<−10° C.) and stirring was continued for another 30 min. The reaction mixture was poured into a saturated aqueous solution of ammonium chloride and the product was extracted into ethyl acetate. The combined organic phases were washed with a saturated aqueous solution of ammonium chloride and brine, dried over sodium sulfate and concentrated under reduced pressure, to give (7α,17β)-7-ethyl-3,17-bis[(trimethylsilyl)oxy]-19-norpregna-3,5-dien-20-yne (158.6 g). The product was used in the following step without further purification.

iii)—A solution of the product obtained in the previous step (15.8.6 g) in acetone (2414 ml) was treated with hydrochloric acid (7 M, 120 ml). After 2 h stirring at room temperature, the reaction mixture was neutralized with a saturated aqueous solution of sodium hydrogencarbonate (965 ml). The acetone was removed under reduced pressure, water was added and the product was extracted into ethyl acetate. The combined organic phases were washed a saturated aqueous solution of ammonium chloride and brine, dried over sodium sulfate and concentrated under reduced pressure, to give (7α,17α)-7-ethyl-17-hydroxy-19-norpregn-4-en-20-yn-3-one (107.6 g). The product was used in the following step without further purification.

iv)—Hydrochloric acid (6 M, 240 ml) was added dropwise to a suspension of dicalite (240 g) in methanol (1200 ml). After 20 min. stirring at room temperature the dicalite was collected by fitration and washed with water until neutral. Then, it was suspended in water (960 ml). With vigorous stirring, copper(II) nitrate trihydrate (145 g) was added, followed by careful addition of a solution of sodium carbonate (72.2 g) in water (360 ml). After 30 min. stirring, the product was collected by filtration and washed with water until neutral. The product was dried at 80° C. under reduced pressure, to give copper(II) carbonate on dicalite (310 g). A mixture of the product obtained under iii (251.7 g) and copper(II) carbonate on dicalite (827 g) in toluene (4470 ml) was heated at reflux temperature for 14 h under removal of water by use of a Dean-Stark trap. The reaction mixture was filtered, the residue thoroughly washed with ethyl acetate (4.5 l), and the filtrate was concentrated under reduced pressure, to give (7α)-7-ethylestr-4-ene-3,17-dione (254.4 g). The product was used in the following step without further purification.

v)—A mixture of the product obtained in the previous step (254.4 g), trimethyl orthoformate (271.6 ml), copper(II) bromide (241.2 g), and methanol (5406 ml) was heated under reflux for 7 h. After cooling, the reaction mixture was filtered. The filtrate was concentrated under reduced pressure and the residu dissolved in ethyl acetate. The resulting solution was washed with a saturated aqueous solution of ammonium chloride, a saturated aqueous solution of sodium hydrogencarbonate and brine, dried over sodium sulfate and concentrated under reduced pressure, to give (7α)-7-ethyl-3-methoxyestra-1,3,5(10)-trien-17-one (208 g). The product was used in the following step without further purification.

vi)—p-Toluenesulfonic acid (9.4 g) was added to a solution of the product obtained in the previous step (208 g) in a mixture of ethylene glycol (317 ml) and triethyl orthoformate (553 ml). The reaction mixture was stirred at room temperature overnight. Then it was poured into ice-water (7885 ml) and stirring was continued for 2 h. Pyridine (80 ml) was added and the mixture was stirred overnight. The resulting precipitate was collected by filtration and dissolved in dichloromethane. The resulting solution was washed with brine, dried over sodium sulfate and concentrated under reduced pressure, to give (7α)-7-ethyl-3-methoxyestra-1,3,5(10)-trien-17-one cyclic 1,2-ethanediyl acetal (224.2 g). The product was used in the following step without further purification.

vii)—A solution of the product obtained in the previous step (224.2 g) and ethylene glycol (380 ml) in dry dimethoxyethane (3229 ml) was treated with pyridinium tribromide (241.0 g). After 4 h stirring a solution of sodium thiosulfate (423.3 g) in water (2510 ml) was added and the product was extracted into ethyl acetate. The combined organic phases were washed with brine, dried over sodium sulfate and concentrated under reduced pressure, to give (7α,16α)-16-bromo-7-ethyl-3-methoxyestra-1,3,5(10)-trien-17-one cyclic 1,2-ethanediyl acetal (303.7 g). The product was used in the following step without further purification.

viii)—A solution of the product obtained in the previous step (303.7 g) in dry dimethyl sulfoxide (2773 ml) was added to a solution of potassium tert-butoxide (343.5 g) in the same solvent (938 ml) and the reaction mixture was stirred at room temperature for 2.5 h. Water (1.5 l) was added the product was extracted into ethyl acetate. The combined organic phases were washed with brine, dried over sodium sulfate and concentrated under reduced pressure, to give (7α)-7-ethyl-3-methoxyestra-1,3,5(10),15-tetraen-17-one cyclic 1,2-ethanediyl acetal (195.5 g). The product was used in the following step without further purification.

ix)—A solution of the product obtained in the previous step (25.0 g) in a mixture of acetone (412 ml) and water (35 ml) was treated with p-toluenesulfonic acid (1.18 g) and the reaction mixture was stirred at room temperature for 1.5 h. A saturated aqueous solution of sodium hydrogencarbonate was added and the product was extracted into ethyl acetate. The combined organic phases were washed with brine, dried over sodium sulfate and concentrated under reduced pressure. Column chromatography afforded (7α)-7-ethyl-3-methoxyestra-1,3,5(10),15-tetraen-17-one (13.0 g).

x)—Following a procedure analogous to that described under i-vi of Example 1, the product obtained in the previous step was converted to (7α,17β)-7-ethyl-3-methoxy-15-methylestra-2,5 (10),14-triene-17-ol.

xi)—Following a procedure analogous to that described under vii of Example 1, the product obtained in the previous step (0.55 g) was converted to (7α,17β)-7-ethyl-17-hydroxy-15-methylestra-4,14-dien-3-one (0.35 g), ¹H NMR (CDCl₃) δ 5.85 (bs, 1H), 3.86 (m, 1H), 1.69 (bs, 3H), 0.97 (s, 3H), 0.90 (t, 3H, J=7.9 Hz).

### EXAMPLE 4

(7α,17β)-7,15-Diethyl-17-hydroxyestra-4,14-dien-3-one.

The title compound was prepared from (7α)-7-ethyl-3-methoxyestra-1,3,5(10),15-tetraen-17-one (Example 3, step ix) following procedures analogous to those described in Example 1. ¹H NMR (CDCl₃) δ 5.84 (bs, IH), 3.84 (m, 1H), 0.97 (t, 3H, J=7.5 Hz), 0.96 (s, 3H), 0.88 (t, 3H, J=7.9 Hz).

### EXAMPLE 5

(7α,17β)-13-Ethyl-17-hydroxy-7,15-dimethylgona-4,14-dien-3-one.

i)—Tetrapropylanmuonium perruthenate (1.3 g) was added to a solution of (7α,17β)-13-ethyl-3-methoxy-7-methylgona-1,3,5(10)-trien-17-ol [FRAD 87961 (1966); 19.5 g] and 4-methylmorpholine N-oxide (21.5 g) in acetone (513 ml). After 30 min. stirring at room temperature the reaction mixture was filtered over dicalite and silica. The filtrate was concentrated under reduced pressure. Column chromatography of the crude product gave (7α)-13-ethyl-3-methoxy-7-methylgona-1,3,5(10)-trien-17-one (11.0 g).

ii)—Following a procedure analogous to that described under ii of Example 1, the product obtained in the previous step (2.9 g) was converted to (7α)-13-ethyl-3-methoxy-7-methyl-17-[(trimethylsilyl)oxy]gona-1,3,5(10),16-tetraene (3.7 g).

iii)—Following a procedure analogous to that described under iii of Example 1, the product obtained in the previous step (3.7 g) was converted to (7α)-13-ethyl-3-methoxy-7-methylgona-1,3,5(10),15-tetraen-17-one (2.5 g).

iv)—Following a procedure analogous to that described under i-vii of Example 1, the product obtained in the previous step was converted to (7α,17β)-13-ethyl-17-hydroxy-7,15-dimethylgona-4,14-dien-3-one, ¹H NMR (CDCl₃) δ 5.85 (bs, 1H), 3.96 (m, 1H), 2.75 (m, 1H), 1.77 (bs, 3H), 0.89 (t, 3H, J 7.6 Hz).

### EXAMPLE 6

(16α,17β)-17-Hydroxy-16-methylestra-4,14-dien-3-one (a) and (16β,17β)-17-hydroxy-16-methylestra-4,14-dien-3-one (b).

i)—Lithium bis(trimethylsilyl)amide (1 M solution in tetrahydrofuran, 7.80 ml) was added dropwise to a solution 3-methoxyestra-1,3,5(10),14-tetraen-17-one [Johnson, W. S. et al, J. Am. Chem. Soc. 79, 2005 (1957); 2.0 g] in a mixture of dry tetrahydrofuran (9.2 ml) and iodomethane, (4.4 ml), cooled to −70° C. The reaction mixture was stirred for 2 h and then treated with another portion of lithium bis(trimetlhylsilyl)amide (1 M solution in tetrahydrofuran, 1.0 ml). After 30 min. stirring, the mixture was poured into a saturated aqueous solution of ammonium chloride and the product was extracted into ethyl acetate. The combined organic phases were washed with brine, dried over sodium sulfate and concentrated under reduced pressure. Column chromatography afforded a mixture of (16α)-3-methoxy-16-methylestra-1,3,5(10),14-tetraen-17-one and (16β)-3-methoxy-16-methylestra-1,3,5(10),14-tetraen-17-one (0.985 g; ratio 1:1), which was used in the following step without further purification.

ii)—Following a procedure analogous to that described under v of Example 1, the mixture obtained in the previous step (0.84 g) was treated with lithium aluminium hydride, to give after chromatography, (16α,17β)-3-methoxy-16-methylestra-1,3,5(10),14-tetraen-17-ol (0.082 g) and (16β,17β)-3-methoxy-16-methylestra-1,3,5(10),14-tetraen-17-ol (0.13 g).

iiia)—Following a procedure analogous to that described under vi of Example 1, (16α,17β)-3-methoxy-16-methylestra-1,3,5(10),14-tetraen-17-ol (0.067 g) was converted to (16α,17β)-3-methoxy-16-methylestra-2,5(10),14-trien-17-ol (0.076 g).

iiib)—Following a procedure analogous to that described under vi of Example 1, (16β,17β)-3-methoxy-16-methylestra-1,3,5(10),14-tetraen-17-ol (0.093 g) was converted to (16β,17β)-3-methoxy-16-methylestra-2,5(10),14-trien-17-ol (0.110 g).

iva)—Following a procedure analogous to that described under vii of Example 1, the product obtained under iiia (0.076 g) was converted to (16α,17β)-17-hydroxy-16-methylestra-4,14-dien-3-one (0.041 g), ¹H NMR (CDCl₃) δ 5.85 (t, 1H, J=1.6 Hz), 5.01 (t, 1H, J=1.6 Hz), 3.41 (d, 1H, J=7.5 Hz), 1.13 (d, 3H, J=7.1 Hz), 1.04 (s, 3H).

ivb)—Following a procedure analogous to that described under vii of Example 1, the product obtained under iiib (0.11 g) was converted to (16β,17β)-17-hydroxy-16-methylestra-4,14-dien-3-one (0.039 g), ¹H NMR (CDCl₃) δ 5.86 (bs, 1H), 5.23 (t, 1H, J=2.8 Hz),4.02(t, 1H, J=7.9 Hz), 1.10(s, 3H), 1.00(d, 3H, J=7.1 Hz).

### EXAMPLE 7

In a manner analogous to the procedures described in Example 2 the following products were prepared:

a)—(7α,16α,17β)-17-Hydroxy-7,16-dimethylestra-4,14-dien-3-one was prepared from (7α)-3-methoxy-7-methylestra-1,3,5(10),14-tetraen-17-one [Segaloff, A. et al, Steroids 22, 99 (1973)]. ¹H NMR (CDCl₃) δ 5.86 (s, 1H), 5.00 (s, 1H), 3.38 (t, 1H, J=7.9 Hz), 1.15 (d, 3H, J=7.1 Hz), 1.03 (s, 3H), 0.84 (d, 3H, J=7.1 Hz).

b)—(7α,16α,17β)-17-Hydroxy-7-ethyl-16-metliylestra-4,14-dien-3-one was prepared from (7α)-3-methoxy-7-ethylestra-1,3,5(10),14-tetraen-17-one (WO 01 05806). ¹H NMR (CDCl₃) δ 5.87 (bs, 1H), 4.93 (t, 1H, J=2.0 Hz), 3.38 (dd, 1H, J=8.3 and 7.1 Hz), 1.15 (d, 3H, J=7.1 Hz), 1.03 (s, 3H), 0.90 (m, 3H).

c)—(7α,16α,17β)-17-Hydroxy-16-methyl-7-vinylestra-4,14-dien-3-one was prepared from (7×)-3-methoxy-7-vinylestra-1,3,5(10),14-tetraen-17-one (WO 01 05806). ¹H NMR (CDCl₃) δ 5.85 (s, IH), 5.78 (m, 1H), 5.14-5.05 (m, 3H), 3.38 (d, 1H, J=8.3 Hz), 2.93 (bs, 1H), 1.13 (d, 3H, J=7.1 Hz), 1.03 (s, 3H).

### EXAMPLE 8

(7α,17β)-7-Ethyl-17-hydroxy-15-methylestra-5(10),14-dien-3-one (a), (3α,7α,17β)-7-ethyl-15-methylestra-5(10),14-diene-3,17-diol (b), and (3β,7α,17β)-7-ethyl-15-methylestra-5 (10),14-diene-3,17-diol (c).

i)—A solution of (7α,17β)-7-ethyl-3-methoxy-15-methylestra-2,5(10),14-triene-17-ol (Example 3, step x; 0.10 g) in a mixture of methanol (0.92 ml) and tetrahydrofuran (0.65 ml) was treated with a solution of oxalic acid (0.32 g) in water (0.54 nm). After 1 h stirring at room temperature, the reaction mixture was poured into water and the product was extracted into ethyl acetate. The combined organic phases were washed with a saturated aqueous solution of sodium hydrogencarbonate, water and brine, dried over sodium sulfate and concentrated under reduced pressure, to give (7α,17β)-7-ethyl-17-hydroxy-15-methylestra-5(10),14-dien-3-one (0.090 g), ¹H NMR (CDCl₃) δ 3.89 (m, 1H), 2.76 (bs, 0.2H), 1.71 (bs, 3H), 0.95 (t, 3H, J=7.5 Hz), 0.94 (s, 3H).

ii)—Sodium borohydride (0.009 g) was added to a solution of the product obtained in the previous step in a mixture of tetrahydrofuran (1.45 ml) and methanol (1.45 ml). The reaction mixture was stirred for 3.5 h and then quenched with acetone. The mixture was poured into water and the product was extracted into ethyl acetate. The combined organic phases were washed with water and brine, dried over sodium sulfate and concentrated under reduced pressure. Column chromatography afforded (3α,7α,17β)-7-ethyl-15-methylestra-5(10),14-diene-3,17-diol (0.035 g), ¹H NMR (CDCl₃) δ 3.88 (dd, 1H, J=9.1 and 8.3 Hz), 3.82 (m, 1H), 1.70 (s, 3H), 1.25 (s, 3H), 0.92 (t, 3H, J=7.1 Hz); and (3β,7α,17β)-7-ethyl-15-methylestra-5(10),14-diene-3,17-diol (0.016 g), ¹H NMR (CDCl₃) δ 4.11 (m, 1H), 3.88 (dd, 1H, J 8.3 and 7.9 Hz), 1.70 (s, 3H), 1.25 (s, 3H), 0.92 (t, 3H, J=7.9 Hz).

### EXAMPLE 9

Biological Results.

The compounds according to the invention were tested for androgenic activity and for progestational activity (the procedures for which have been described above) and rated according to the following scheme:

| | Androgenic | | |
|---|---|---|---|
| Example | activity | Progestational activity | Ratio A/P activity |
| 1 | ++ | ++ | 1:1 |
| 2 | + | ++ | 1:5 |
| 3 | ++ | + | 16:1 |
| 4 | + | + | 1:1 |
| 5 | ++ | + | 2:1 |
| 6a | + | + | 5:1 |
| 6b | ++ | + | 10:1 |
| 7a | ++ | + | 3:1 |
| 7b | ++ | + | 17:1 |
| 7c | ++ | + | 14:1 |
| 8a | Prodrug | Prodrug | |
| 8b | Prodrug | Prodrug | |
| 8c | Prodrug | Prodrug | |
| Reference | +++ | − | 150:1 |
| compound | | | |
| (Segaloff | | | |
| comp.*) | | | |

(−) very weak activity found;
(+) activity found;
(++) high activity;
(+++) excellent activity.
*(7α,17β)-7-methyl-17-hydroxyestra-4,14-dien-3-one

1. A compound satisfying the general formula I given below 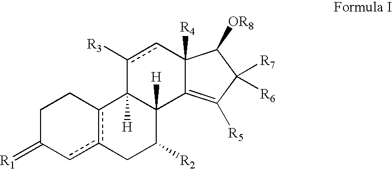
wherein
R₁ is O, (H,H), (H,OR), NOR, with R being hydrogen, (C₁₋₆) alkyl, or (C₁₋₆) acyl;
R₂ is hydrogen, (C₁₋₄) alkyl, or (C₂₋₄) alkenyl;
R₃ is hydrogen, (C₁₋₄) alkyl, or (C₂₋₄) alkenyl;
R₄ is (C₁₋₂) alkyl;
R₅ is hydrogen, (C₁₋₄) alkyl, (C₂₋₄) alkenyl;
R₆ and R₇ are independently hydrogen, (C₁₋₄) alkyl, or (C₂₋₄) alkenyl;
R₈ is hydrogen, or (C₁₋₁₅) acyl;
and the dotted lines indicate optional bonds, and wherein at least one of R₅, R₆ and R₇ is not hydrogen.

2. A compound according to claim 1, characterized in that R₃ is hydrogen or β-methyl.

3. A compound according to claim 2, characterised in that R₄ is methyl.

4. A compound according to anyone of claim 1-3, characterized in that R₁ is oxo, R₃ is hydrogen, R₄ is methyl or ethyl, and the dotted lines indicate a Δ⁴ double bond.

5. A compound according to anyone of claim 1-4, characterised in that R₂ is selected from the group consisting of methyl, ethyl, and ethenyl, either or both of R₅ and R₆ are methyl and R₇ is hydrogen.

6. A compound according to claim 4 or 5, characterized in that R₅ or R₆ is methyl.

7. A compound according to claim 6, characterised in that R₆ is methyl.

8. A compound according to claim 7, characterised in that R₂ is methyl, ethyl or vinyl.

9. The compound (7α,17β)-17-hydroxy-7,15-dimethylestra-4,14-dien-3-one or (7α,16α,17β)-17-hydroxy-7,16-dimethylestra-4,14-dien-3-one.

10. A compound according to any one of the preceding claims for use as a medicine.

11. A pharmaceutical composition comprising a pharmaceutically acceptable carrier and comprising, as a medicinally active agent, a steroid compound satisfying the general formula I as described in the specification.

12. The use of a compound according to any one of the claims 1-9, for the preparation of a medicine in the therapy of an insufficiency selected from the group consisting of androgen-insufficiency, progestagen-insufficiency, and combined androgen-progestagen insufficiency.

13. A use according to claim 12, characterized in that the therapy comprises the combined administration of a compound according to any one of the claims 1-9 and an androgen that is not 5α-reducable, such as 7α-methyl nandrolone.

14. A pharmaceutical composition comprising a pharmaceutically acceptable carrier and comprising, as a medicinally active agent, a compound according to any one of the claims 1-9.

15. A kit for male contraception comprising means for the administration of a progestagen and means for the administration of an androgen, characterised in that either or both of the means is a pharmaceutical composition according to claim 11.

16. A kit according to claim 15, characterised in that it comprises means for the administration of a compound according to any one of the claims 1-9 in combination with an androgen.

17. A kit according to claim 15, characterised in that it comprises means for the administration of a compound according to any one of the claims 1-9 in combination with a progestagen.
